(19) United States
(12) Patent Application Publication
(10) Pub. No.: US 2020/0268987 A1
(43) Pub. Date: Aug. 27, 2020

(54) Cannula Assembly for Higher Viscosity Injectable Drugs

(71) Applicant: Minhong Yu, Moorpark, CA (US)

(72) Inventor: Minhong Yu, Moorpark, CA (US)

(21) Appl. No.: 16/798,540

(22) Filed: Feb. 24, 2020

Related U.S. Application Data
(60) Provisional application No. 62/841,389, filed on May 1, 2019.
(60) Provisional application No. 62/810,384, filed on Feb. 26, 2019.

(51) Int. Cl. A61M 5/32; A61M 5/31; A61M 5/34

(52) U.S. Cl. CPC A61M 5/3286; A61M 5/3134; A61M 2207/10; A61M 5/34; A61M 2205/0222; A61M 5/3293

(57) ABSTRACT

A cannula needle for reducing injection force in connection with high viscosity fluid medications or medications with wide viscosity ranges. The cannula needle includes a skin penetrating segment, an enlarged rear end segment, and a transition segment disposed between the skin penetrating segment and the enlarged rear end segment. The skin penetrating segment includes a substantially uniform outer diameter and a substantially uniform inner diameter for patient comfort. The enlarged rear end segment has an inner diameter that is greater than the inner diameter of the skin penetrating segment. The transition segment includes a funnel shape for fluidly connects the inner diameter of the enlarged rear end segment and the inner diameter of the skin penetrating segment.

### CROSS-REFERENCE TO RELATED APPLICATIONS

This application claims priority as a non-provisional to U.S. Provisional Application Ser. No. 62/810,384 filed Feb. 26, 2019, entitled “Novel Needle Cannula Design for Higher Viscosity Drug Product,” and as a non-provisional to U.S. Provisional Application Ser. No. 62/841,389 filed May 1, 2019, entitled “Needle Cannula Design for Higher Viscosity Drug Products,” the contents of both being incorporated herein by reference in their entireties.

### FIELD

The present disclosure relates generally to cannula assemblies for reducing the injection force experienced when trying to deliver flowable medications with higher viscosities. More particularly, the present disclosure relates to cannula assemblies for reducing the injection force while also providing a skin penetrating needle portion that is consistent with the various gauge options of traditional staked needles.

### BACKGROUND

More and more injectable drugs being developed have high viscosities due to various factors such as formulation composition, concentration, etc. Whether the injection is via manual injection or via auto-injectors, the high viscosity of the medication proves a challenge during injection from a needle. In this regard, human hands can only exert a specific amount of force while maintaining steady injection. Similarly, auto-injectors are mostly spring based automatic injector devices wherein the spring can only deliver a specific force. In addition, per regulatory requirements, most auto-injectors need to deliver all the medication under a prescribed time such as 15 seconds. As such, higher viscosity drugs prove a challenge during auto-injection.

Many injectable drugs also have a wide viscosity range depending on the actual pH of the medication within the allowed pH range, concentration, temperature, etc. This wide viscosity range proves a challenge for injection as, at the low end of the spectrum (i.e., drug with a low viscosity), injection may prove too fast while, at the other end of the spectrum (i.e., drug with a high viscosity), injection may prove to be too slow.

To overcome the challenges with respect to injecting drugs of high viscosities, a tapered needle design that begins at one end with a larger diameter followed by a gradual reduction in diameter to the desired needle diameter at the needle tip has been developed. These types of needle are often referred to as a tapered needle. While these needles do prove effective in reducing injection force for high viscosity drugs, these needles also create injection pain problems as only the tip of the needle is at the desired outer diameter. As the needle penetrates the patient's skin, the needle diameter increases, which results in expansion of the injection wound and more pain/discomfort to the patient than a standard needle.

What is needed therefor is a needle design that promotes a lower injection force needed for injecting drugs of higher viscosities while also promoting consistency in injecting drugs of varying viscosities.

### SUMMARY

The above and other needs are met by the present disclosure, which provides in one embodiment a cannula assembly having a syringe barrel, a syringe hub, and a needle. The syringe barrel defines a chamber that extends from a first end to a second end. The syringe hub is disposed adjacent the second end of the chamber. The needle includes a skin penetrating segment, an enlarged rear end segment, and a transition segment. The skin penetrating segment has a substantially uniform outer diameter and a substantially uniform inner diameter. The enlarged rear end segment has an inner diameter that is greater than the inner diameter of the skin penetrating segment and at least a portion of the enlarged rear end segment is disposed within the syringe hub such that the needle is in fluid communication with the chamber of the syringe barrel. The transition segment is disposed between the skin penetrating segment and the enlarged rear end segment for fluidly connecting the inner diameter of the enlarged rear end segment and the inner diameter of the skin penetrating segment. The transition segment includes a funnel shaped inner diameter.

According to certain embodiments, the second end of the chamber includes a funnel shape sloped towards the enlarged rear end segment of the needle.

According to certain embodiments, the enlarged rear end segment includes a substantially uniform inner diameter and a substantially uniform outer diameter.

According to certain embodiments, the inner diameter of the enlarged rear end segment is funnel shaped.

According to certain embodiments, the skin penetrating segment includes a length ranging from about 5 mm to about 14 mm and the substantially uniform inner diameter includes a diameter ranging from about 0.108 mm to about 0.26 mm.

According to certain embodiments, the needle includes an inner wall extending from the skin penetrating segment through the enlarged rear end segment wherein the inner wall includes a hydrophilic coating.

According to certain embodiments, the needle includes an inner wall extending from the skin penetrating segment through the enlarged rear end segment wherein the inner wall includes patterned grooves disposed along a length of at least a portion of the inner wall.

According to certain embodiments, the skin penetrating segment includes an outer wall wherein the outer wall includes patterned protrusions extending along a length of at least a portion of the outer wall to assist in skin penetration.

According to certain embodiments, the transition segment and the enlarged rear end segment are disposed within the syringe hub. According to other embodiments, the enlarged rear end segment is disposed within the syringe hub and the transition segment is disposed immediately adjacent the syringe hub opposite the chamber.

According to another embodiment of the present disclosure, a cannula needle is provided that includes a skin penetrating segment, an enlarged rear end segment, and a transition segment. The skin penetrating segment includes a substantially uniform outer diameter and a substantially uniform inner diameter. The enlarged rear end segment has an inner diameter that is greater than the inner diameter of the skin penetrating segment. The enlarged rear end segment is dimensioned and configured to be disposed within a syringe hub for fluidly connecting the cannula needle to a cannula assembly. The transition segment is disposed between the skin penetrating segment and the enlarged rear end segment for fluidly connecting the inner diameter of the enlarged rear end segment and the inner diameter of the skin penetrating segment. The transition segment includes a funnel shaped inner diameter.

According to certain embodiments, the enlarged rear end segment includes a substantially uniform inner diameter and a substantially uniform outer diameter.

According to certain embodiments, the inner diameter of the enlarged rear end segment is funnel shaped.

According to certain embodiments, the skin penetrating segment includes a length ranging from about 5 mm to about 14 mm and the substantially uniform inner diameter includes a diameter ranging from 0.108 mm to about 0.26 mm.

According to certain embodiments, the needle includes an inner wall extending from the skin penetrating segment through the enlarged rear end segment wherein the inner wall including a hydrophilic coating.

According to certain embodiments, the needle includes an inner wall extending from the skin penetrating segment through the enlarged rear end segment wherein the inner wall includes patterned grooves disposed along a length of at least a portion of the inner wall.

According to certain embodiments, the skin penetrating segment includes an outer wall wherein the outer wall includes patterned protrusions extending along a length of at least a portion of the outer wall to assist in skin penetration.

According to certain embodiments, a method of forming the cannula needle of includes: providing a metal tube having an inner diameter corresponding substantially to the inner diameter of the enlarged rear end segment; inserting a sizing pin into a portion of the metal tube; rotating the metal tube with the sizing pin inserted; and advancing a forming die against the portion of the metal tube within the sizing pin inserted thereto to form the skin penetrating segment.

According to certain embodiments, a method of forming the cannula needle includes providing a metal tube having an inner diameter corresponding substantially to the inner diameter of the enlarged rear end segment; securing the enlarged rear end segment to a fixture such that a second portion of the metal tube extends from the fixture; and pulling the second portion of the metal tube away from the fixture to form the skin penetrating segment.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a partial cross-sectional view of a cannula assembly according to one embodiment of the disclosure;

FIG. 2 is a cross-sectional view of a needle portion of a cannula assembly according to one embodiment of the disclosure;

FIG. 3 shows a comparison of the average injection force for a 30 cP glycerol mimic prefilled syringe installed with a standard needle as compared to three different needles of the present disclosure;

FIG. 4 shows a comparison of the average injection force for a 200 cP glycerol mimic prefilled syringe installed with a standard needle as compared to three different needles of the present disclosure;

FIG. 5 shows a comparison of the average injection force for a 100 cP glycerol mimic being injected through a standard 1 ml long prefilled syringe installed with a standard needle as compared to a needle design of the present disclosure;

FIG. 6 shows a comparison of the average injection force for a 100 cP glycerol mimic being injected through a standard 2.25 ml long prefilled syringe installed with a standard needle as compared to a needle design of the present disclosure;

FIGS. 7A-7D are cross-sectional views of various inner needle wall patterns according to certain embodiments of the disclosure;

FIG. 8 is a cross-sectional view of a needle having a patterned external wall and a substantially uniform inner needle wall according to one embodiment of the disclosure; and

FIG. 9 is a representative figure depicting an exemplary process for the formation of a needle design of the present disclosure.

### DETAILED DESCRIPTION

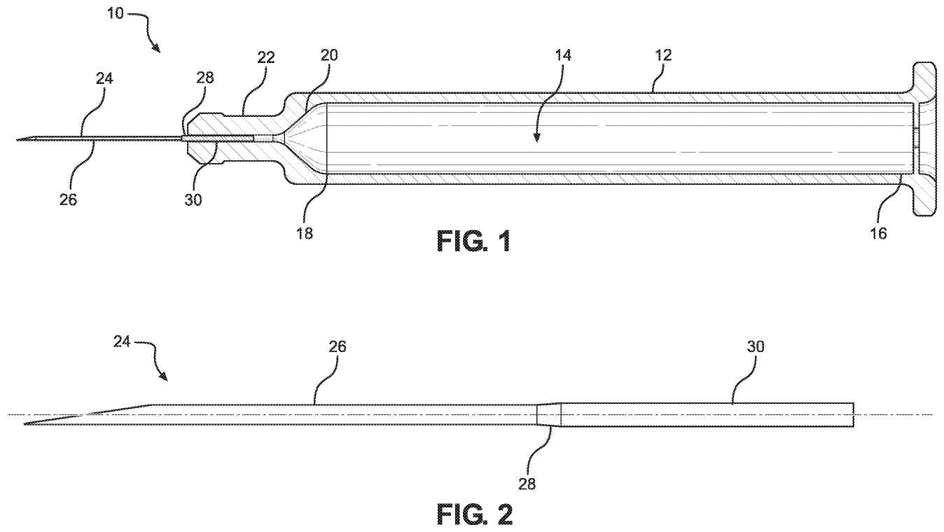

Referring to FIG. 1, a cannula assembly 10 according to one embodiment of the present disclosure broadly includes a syringe barrel 12 defining a chamber 14 configured for receiving a flowable medication. The chamber 14 extends from a first end 16 to a second end 18. The cannula assembly 10 further includes a syringe hub 22 adjacent the second end 18 of the chamber 14 and a needle 24 secured within the syringe hub 22 such that the needle 24 is in fluid communication with the chamber 14. In operation, the leading end of the needle 24 is configured to be inserted into the patient's skin while a plunger (not shown) is operable to be advanced through the chamber 14 from the first end 16 to the second end 18 to advance the flowable medication through the needle 24 for injection into the patient. While the concepts of the present disclosure are shown and described in reference to a cannula assembly 10 with a staked needle, it should be understood that the features of the present disclosure could be incorporated into other needle related assemblies such as auto-injectors, attachable needles (e.g., luer-lock syringes), etc.

Referring to FIG. 2, the needle 24 preferably includes a skin penetrating segment 26, an enlarged rear end segment 30, and a transition segment 28 disposed between the skin penetrating segment 26 and the enlarged rear end segment 30. The skin penetrating segment 26 preferably includes a substantially uniform outer diameter and a substantially uniform inner diameter that is consistent with the various gauge options of traditional staked needles. In preferred embodiments, the skin penetrating segment 26 is between about 5 mm and about 14 mm in length, and specific preferred length generally varies based on the particular medication characteristics that is being injected through the needle and/or the depth in which the medication is intended to be injected. The uniform outer diameter and uniform inner diameter of the skin penetrating segment are intended to prevent additional pain and discomfort to the patient during injection as compared to skin penetrating segments having larger or varying diameters. In other words, the skin penetrating segment 26 of the present needle assembly is dimensioned and configured to be of substantially the dimensions and configuration of standard cannula gauges to ensure the same needle skin penetration experience as standard needles. The cannula assembly 10 of the embodiments described herein is believed to be particularly advantageous when skin penetrating segments 26 have internal and external diameters corresponding to smaller gauge needles as needles with smaller external diameters are able to be used as a result of the injection pain reduction of the needle 24 design of the present disclosure. For example, for drug viscosities that would typically require a 27-gauge needle, a needle 24 having a smaller gauge skin penetrating segment 26 than the standard 27-gauge needle may be used to reduce patient discomfort. Additionally, needles 24 having shorter skin penetrating segments 26 than standard needles may be utilized as a result of the injection force reduction of the present disclosure. As a result of the shorter length, lower shearing stress to the protein of biological drug occurs during the injections. Thus, the susceptibility risk of protein to unfold and aggregate is reduced.

With continued reference to FIG. 2, the enlarged rear end segment 30 includes an inner diameter that is larger than the respective inner diameter of the skin penetrating segment 26. In preferred embodiments, the rear end segment includes at least a uniform outer diameter to allow easier connection of the needle 24 to the hub 22. In certain embodiments, the enlarged rear end segment 30 may also include a substantially uniform inner diameter that is larger than the respective inner diameter of the skin penetrating segment 26. More specifically, according to preferred embodiments, the substantially uniform inner diameter of the skin penetrating segment 26 includes a diameter ranging from about 0.108 mm to about 0.26 mm, and the enlarged rear end segment 30 preferably includes a substantially uniform inner diameter that is between about 25% and about 600% larger, and most preferably about 200%-500% larger, than the corresponding inner diameter of the skin penetrating segment 26. In other embodiments, the enlarged rear end segment 30 may include a sloped inner diameter (e.g., funnel or conical shaped) such that the rear end segment 30 includes a larger inner diameter adjacent the connection to the syringe barrel 12 and a smaller inner diameter adjacent to the skin penetrating segment 26. In certain embodiments in which the inner diameter of the rear end segment 30 is sloped, the transition segment 28 described further below may be omitted as it is incorporated into the slope/funnel shape of the rear end segment 30.

In embodiments in which the enlarged rear end segment 30 includes a substantially uniform inner diameter (or otherwise a very gentle slope), the transition segment 28 is provided to transition the inner diameter of the needle 24 from the enlarged inner diameter of the rear end segment 30 to the smaller inner diameter of the skin penetrating segment 26. The transition segment 28 is important to provide a laminar flow throughout the entirety of needle 24, which reduces the injection force needed for the cannula assembly 10.

In preferred embodiments, the transition segment 28 is funnel or conically shaped as shown. In certain embodiments, the funnel shape is only internal to the transition segment 28. According to this embodiment, the outer diameter of the transition segment may be uniform (e.g., the same as the outer diameter of the rear end segment 30 ). In other embodiments, both the inner diameter and outer diameter of the transition segment 28 are sloped as shown in FIG. 2. Also, in most preferred embodiments, the inner diameter transition segment 28 slopes inward at an angle of between about 1° and 20°, and most preferably between about 2° and 10°. The length of the transition segment 28 will then vary depending on the angle of the transition segment 28 and the difference in size between the inner diameter of the enlarged rear end segment 30 and the inner diameter of the skin penetrating segment 26. In other words, when comparing the transition segment of two different needles 24 having the same inner diameters of the rear end segment 30 and skin penetrating segment 26 but different sloped transition segments 28, the smaller angled transition segment 28 would require a longer transition segment 28 while the larger angled transition segment 28 would allow for a shorter transition segment 28.

Referring back to FIG. 1, at least a portion of the enlarged rear end segment 30 is disposed within the hub 22 to secure the needle 24 to the cannula assembly 10 while leaving the skin penetrating segment 26 exposed for inserting into the skin of the patient. To secure the needle 24 to the hub 22, the rear end segment 30 may be glued (such as with UV cured glue) to an aperture disposed within the hub 22. It should be understood that the aperture of the hub 22 will be dimensioned and configured based on the size and shape of the outer diameter of the enlarged rear end segment 30 that the hub 22 aperture is intended to receive. In preferred embodiments, the entirety of the enlarged rear end segment 30 is secured within the hub 22 aperture. The transition segment 28 is then preferably disposed just outside the hub 22 to prevent any penetration into the skin of segment 28 (given the length of the skin penetrating segment 26) while also allowing the aperture of the hub 22 to be formed with a uniform diameter to receive a rear end segment 30 having a uniform outer diameter. Positioning the transition segment 28 just outside the hub 22 also allows the transition segment 28 to be covered by the standard glue bulge at the tip of the syringe hub 22. In alternate embodiments, the transition segment 28 may be disposed fully (or partially) within the hub 22.

According to preferred embodiments, and with continued reference to FIG. 1, the second end 18 of chamber 14 of the syringe barrel 12 may also include a syringe barrel transition segment 20 to further assist in reducing injection force in combination with the stepped-down needle 24. According to this embodiment, the second end of chamber 18 is formed (such as with forming pins, dies, mold shapes, etc. depending on the particular process used to form the syringe barrel 12) to include a funnel shape leading to the enlarged rear end segment 30. In certain embodiments, and particularly when the syringe barrel 12 and syringe hub 22 are formed of two separate parts intended to connect to each other, a portion of the syringe barrel transition segment 20 may be included in the syringe hub 22 aperture. In other words, according to this embodiment, a first portion of the funnel shape of the transition segment 20 is formed as part of the second end 18 of the syringe barrel chamber 14 while the remaining portion of the funnel shape is formed as the part of the syringe hub 22 aperture that is intended to receive the rear end segment 30 of needle 24. The optimal syringe funnel geometry of transition segment 20 is dependent on the size of the inner diameters of rear end segment 30 and skin penetrating segment 26 as well as general fluid dynamic principles such as the Hagen-Poiseuille relation.

In operation, the syringe barrel transition segment 20 reduces the injection resistance and turbulence of the flowable medication as the medication flows from the chamber 14 to the enlarged rear end segment 30 of the needle 24. The larger the inner diameter of the transition segment 20 as it transitions from the larger inner diameter at the second end 18 of chamber 20 to the smaller inner diameter at the connection point of the rear end segment 30, the greater the reduction in injection resistance and turbulence. Further, increasing the inner diameter size of the transition segment 20 of syringe barrel 12 and rear end segment 30 of needle 24 will not impact patient comfort compared to needles of standard gauges because only the skin penetrating segment 26 of needle 24 is intended to be inserted into the patient.

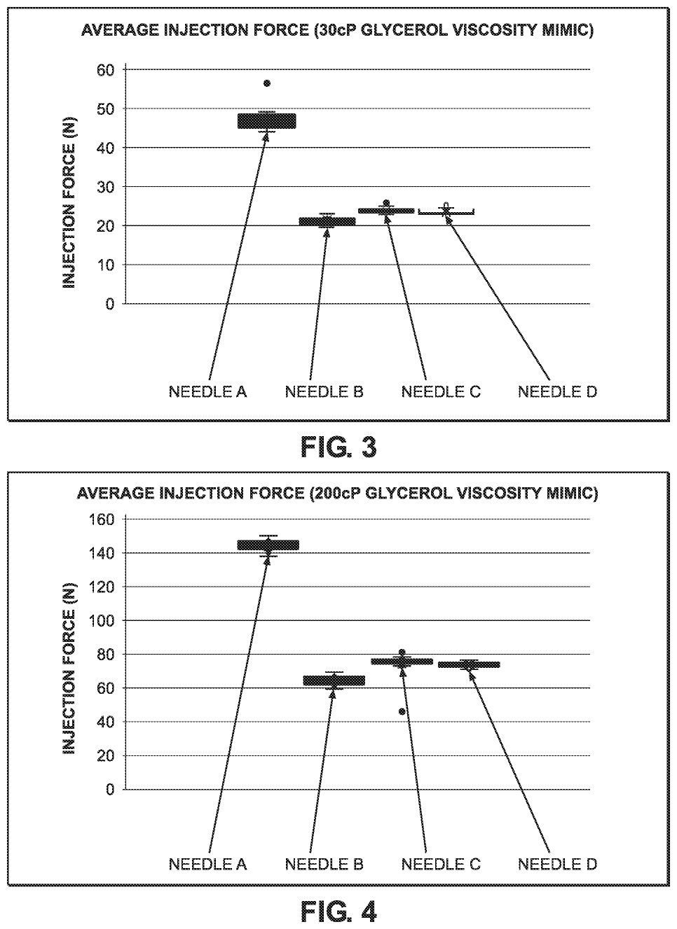

Referring to FIG. 3, results are provided comparing the average injection force of a 30 cP viscosity glycerol mimic being injected through a standard 2.25 mL glass prefilled syringe. In this example, four needles were tested. Needle A was a standard thin wall 27 G needle. Needle B, C, and D included a needle design along the lines of FIG. 2 of the present disclosure with the following dimensions:

Needle B

| | |
|---|---|
| Enlarged rear end segment: | Inner diameter - .608 mm |
| | Length - 9 mm |
| Transition segment: | Inner diameter slope angle - 1° |
| | Length - 3 mm |
| Skin penetrating segment: | Inner diameter - .27 mm |
| | Length - 8 mm |

Needle C

| | |
|---|---|
| Enlarged rear end segment: | Inner diameter - .4 mm |
| | Length - 8 mm |
| Transition segment: | Inner diameter slope angle - 5° |
| | Length - .57 mm |
| Skin penetrating segment: | Inner diameter - .31 mm |
| | Length - 10.93 mm |

Needle D

| | |
|---|---|
| Enlarged rear end segment: | Inner diameter - .4 mm |
| | Length - 8 mm |
| Transition segment: | Inner diameter slope angle - 10° |
| | Length - .28 mm |
| Skin penetrating segment: | Inner diameter - .31 mm |
| | Length - 11.22 mm |

As shown in FIG. 3, use of each of Needles B, C, and D incorporating the features of needle 24 of the present disclosure required a lower injection force than the standard thin wall 27 G needle.

Referring to FIG. 4, similar test results are shown comparing the average injection force of a 200 cp viscosity glycerol mimic being injected through a standard 2.25 mL glass prefilled syringe with a standard 27 G thin wall needle (Needle A) to the same 200 cp viscosity glycerol mimic being injected through a standard 2.25 mL glass prefilled syringe with the Needle B, Needle C, and Needle D designs as described above.

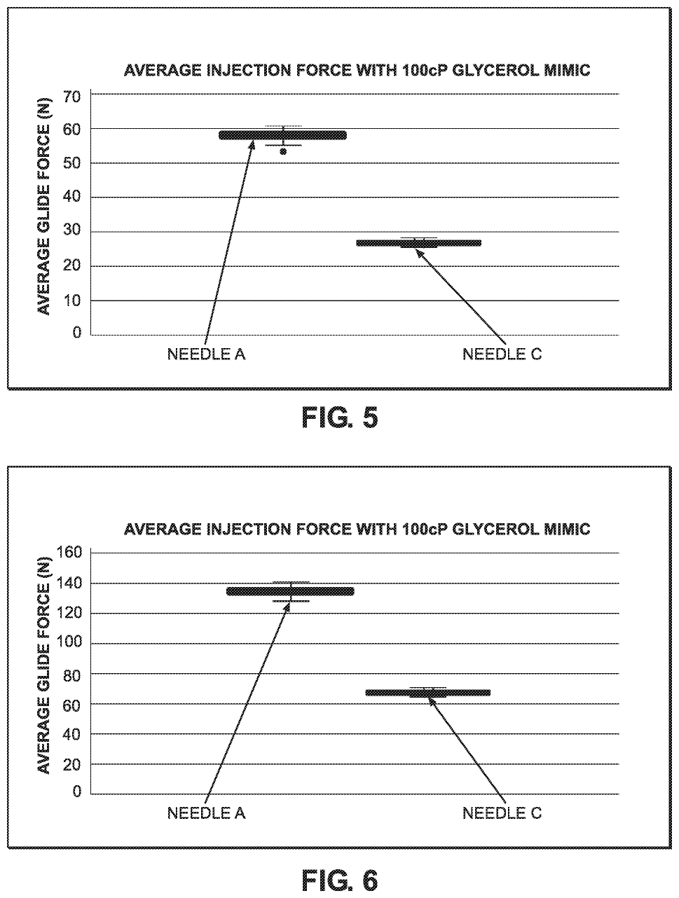

Referring to FIG. 5, similar test results are shown comparing the average injection force of a 100 cP viscosity glycerol mimic being injected through a standard 1 ml long glass prefilled syringe with a standard thin wall 27 G needle (Needle A) to the same 100 cP viscosity glycerol mimic being injected through a standard 1 ml long glass prefilled syringe with Needle C described above.

Referring to FIG. 6, similar test results are again shown comparing the average injection force of 100 cP viscosity glycerol mimic being injected through a standard 2.25 ml glass prefilled syringe with a standard thin wall 27 G needle (Needle A) to the same 100 cP viscosity glycerol mimic being injected through a standard 2.25 ml glass prefilled syringe with Needle C described above.

It should be understood that the cannula assembly 10 of the present disclosure is not limited to the design as shown in FIGS. 1-2. Other designs are contemplated utilizing the principle that the rear end segment 30 connected to the hub 22 includes an enlarged inner diameter as compared to the uniform inner diameter of the skin penetrating segment 26. For example, the needle assembly could include multiple rear end segments 30 and/or transitioning segments 26 to provide multiple “step downs” in diameter when transitioning the inner diameter needle size from the portions disposed within the hub 22 to the skin penetrating segment 26.

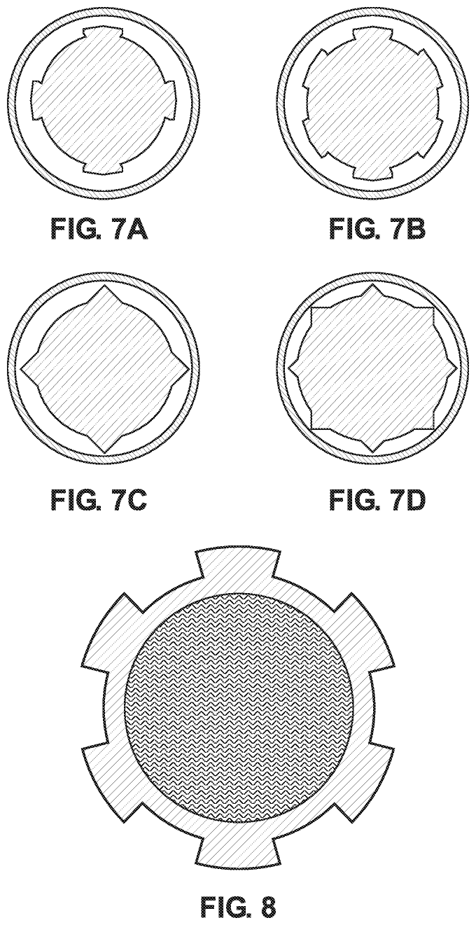

According to another aspect of the disclosure, the inner diameter of the various segments of the needle 24 could include patterns or grooves to further facilitate fluid flow and reduce the injection force needed for injecting the medication of the needle assembly 10. In particular, the inner diameter could include certain portions that extend out from the radial center of the needle further than other portions along the same cross-sectional plane to further facilitate fluid flow and reduce injection force. Cross sections of exemplary inner diameter patterns are depicted in FIGS. 7A-7D. It should be understood that the patterned inner diameter can be included throughout the length of the needle 24 or only in certain portions. For example, according to certain embodiments, only the enlarged rear end segment 30 includes patterned grooves. In other embodiments, only the skin penetrating segment 26 includes the particular pattern. The grooves/patterns may be formed by a stretching or broaching process.

According to another aspect of the disclosure, the outer diameter of the skin penetrating segment 26 could include patterns or grooves to facilitate easier skin penetration, particularly near the skin penetrating end of the skin penetrating segment 26. A cross section of one exemplary skin penetrating segment 26 with a patterned outer diameter and smooth inner diameter is depicted in FIG. 8. It should also be understood that certain skin penetrating segments 26 could include both a patterned inner diameter and a patterned outer diameter along at least a portion of the skin penetrating segment.

According to another aspect of the disclosure, a lubricating agent (such as a hydrophilic, silicone, or Teflon coating) is applied along the inner diameter of the needle 24 to facilitate fluid flow and further reduce injection force.

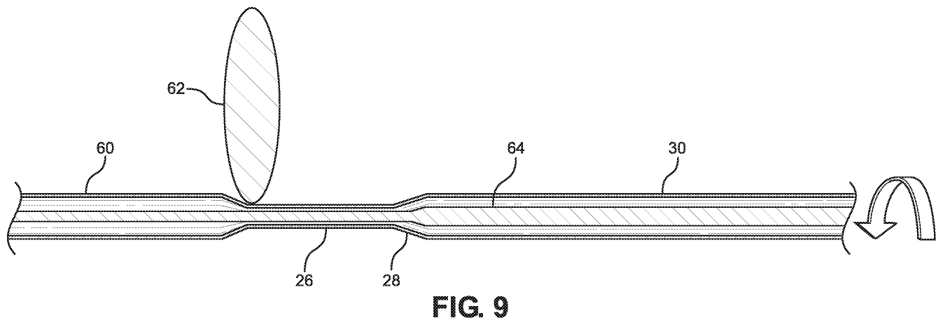

The needle assembly of the present disclosure can be formed by various machining processes. Referring to FIG. 9, one exemplary process is via tube spinning in which a sizing pin 64 is inserted into a metal tube 60 having an inner diameter corresponding to the desired inner diameter of the enlarged rear end segment 30. The metal tube is rotated while a forming die 62 is pushed against the exterior surface of the metal tube 60 along the intended skin penetrating segment 26 to form the smaller diameter skin penetrating segment 26. The metal tube 60 may also be heated during this process to aid in the forming by forming die 62 along the length of the skin penetrating segment. It should be understood that the size of the inner diameter of the skin penetrating segment 26 is dependent on the depth in which the forming die 62 is pressed against the exterior surface of the metal tube 60. The transition segment 28 may be formed in a similar manner by advancing the forming die 62 along the length of the transition segment 28 at an increasing depth as the forming die 62 moves from the rear end segment 30 to the skin penetrating segment 26 (or as the tube 60 traverses the forming die 62) until the depth remains constant along the length of the skin penetrating segment 26.

Another exemplary process for forming the skin penetrating segment 26 includes starting with a metal tube the size of the enlarged rear end segment 30, attaching the rear end segment 30 to a fixture, and then elongating the skin penetrating segment 26 extending from the fixture such that the skin penetrating segment 26 includes a smaller inner/outer diameter as a result of the pulling motion.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A cannula assembly comprising:
a syringe barrel defining a chamber, the chamber extending from a first end to a second end;
a syringe hub disposed adjacent the second end of the chamber; and
a needle including:
a skin penetrating segment having a substantially uniform outer diameter and a substantially uniform inner diameter,
an enlarged rear end segment having an inner diameter that is greater than the inner diameter of the skin penetrating segment, at least a portion of the enlarged rear end segment being disposed within the syringe hub such that the needle is in fluid communication with the chamber of the syringe barrel, and
a transition segment disposed between the skin penetrating segment and the enlarged rear end segment for fluidly connecting the inner diameter of the enlarged rear end segment and the inner diameter of the skin penetrating segment, the transition segment including a funnel shaped inner diameter.

2. The cannula assembly of claim 1 wherein the second end of the chamber includes a funnel shape sloped towards the enlarged rear end segment of the needle.

3. The cannula assembly of claim 1 wherein the enlarged rear end segment includes a substantially uniform inner diameter and a substantially uniform outer diameter.

4. The cannula assembly of claim 1 wherein the inner diameter of the enlarged rear end segment is funnel shaped.

5. The cannula assembly of claim 1 wherein the skin penetrating segment includes a length ranging from about 5 mm to about 14 mm and the substantially uniform inner diameter includes a diameter ranging from about 0.108 mm to about 0.26 mm.

6. The cannula assembly of claim 1 wherein the needle includes an inner wall extending from the skin penetrating segment through the enlarged rear end segment, the inner wall including a hydrophilic coating.

7. The cannula assembly of claim 1 wherein the needle includes an inner wall extending from the skin penetrating segment through the enlarged rear end segment and wherein the inner wall includes patterned grooves disposed along a length of at least a portion of the inner wall.

8. The cannula assembly of claim 1 wherein the skin penetrating segment includes an outer wall and wherein the outer wall includes patterned protrusions extending along a length of at least a portion of the outer wall to assist in skin penetration.

9. The cannula assembly of claim 1 wherein the transition segment and the enlarged rear end segment are disposed within the syringe hub.

10. The cannula assembly of claim 1 wherein the enlarged rear end segment is disposed within the syringe hub and the transition segment is disposed immediately adjacent the syringe hub opposite the chamber.

11. A cannula needle comprising:
a skin penetrating segment having a substantially uniform outer diameter and a substantially uniform inner diameter;
an enlarged rear end segment having an inner diameter that is greater than the inner diameter of the skin penetrating segment, the enlarged rear end segment being dimensioned and configured to be disposed within a syringe hub for fluidly connecting the cannula needle to a cannula assembly; and
a transition segment disposed between the skin penetrating segment and the enlarged rear end segment for fluidly connecting the inner diameter of the enlarged rear end segment and the inner diameter of the skin penetrating segment, the transition segment including a funnel shaped inner diameter.

12. The cannula needle of claim 11 wherein the enlarged rear end segment includes a substantially uniform inner diameter and a substantially uniform outer diameter.

13. The cannula needle of claim 11 wherein the inner diameter of the enlarged rear end segment is funnel shaped.

14. The cannula needle of claim 11 wherein the skin penetrating segment includes a length ranging from about 5 mm to about 14 mm and the substantially uniform inner diameter includes a diameter ranging from 0.108 mm to about 0.26 mm.

15. The cannula needle of claim 11 wherein the needle includes an inner wall extending from the skin penetrating segment through the enlarged rear end segment, the inner wall including a hydrophilic coating.

16. The cannula needle of claim 11 wherein the needle includes an inner wall extending from the skin penetrating segment through the enlarged rear end segment and wherein the inner wall includes patterned grooves disposed along a length of at least a portion of the inner wall.

17. The cannula needle of claim 11 wherein the skin penetrating segment includes an outer wall and wherein the outer wall includes patterned protrusions extending along a length of at least a portion of the outer wall to assist in skin penetration.

18. A method of forming the cannula needle of claim 11, the method comprising:
providing a metal tube having an inner diameter corresponding substantially to the inner diameter of the enlarged rear end segment;
inserting a sizing pin into a portion of the metal tube;
rotating the metal tube with the sizing pin inserted; and
advancing a forming die against the portion of the metal tube within the sizing pin inserted thereto to form the skin penetrating segment.

19. A method of forming the cannula needle of claim 1, the method comprising:
providing a metal tube having an inner diameter corresponding substantially to the inner diameter of the enlarged rear end segment;
securing the enlarged rear end segment to a fixture such that a second portion of the metal tube extends from the fixture; and
pulling the second portion of the metal tube away from the fixture to form the skin penetrating segment.
